(19) United States
(12) Patent Application Publication
(10) Pub. No.: US 2004/0249187 A1
(43) Pub. Date: Dec. 9, 2004

(54) Method for producing chiral amino acid derivatives

(76) Inventors: Joachim Rudolph, Guilford, CT (US); Frithjof Hannig, Munster (DE)

Correspondence Address: LANXESS CORPORATION, PATENT DEPARTMENT/ BLDG 14, 100 BAYER ROAD, PITTSBURGH, PA, 15205-9741, US

(21) Appl. No.: 10/481,499

(22) PCT Filed: Jun. 6, 2002
(86) PCT No.: PCT/EP02/06205

(30) Foreign Application Priority Data
Jun. 19, 2001 (DE) 101 29 510.3

(51) Int. Cl. C07C 229/02

(52) U.S. Cl. 560/39; 560/169

(57) ABSTRACT

The invention relates to a method for producing chiral amino acid derivatives, characterised in that free carboxylic acid groups in an amino acid derivative are first converted into nitro ketones, and said nitro ketones are then converted into the corresponding nitro alcohols and amino alcohols by means of reduction. The invention also relates to the nitro ketones and nitro alcohols obtained as intermediate products.

The invention relates to a process for preparing chiral amino acid derivatives and novel intermediates.

Derivatives of nonproteinogenic amino acids, for example (2S,4R)-4-hydroxy-omithine and its homologue (2S,5R)-5-hydroxylysine have been the subject of numerous synthesis attempts. The former are commercially promising, for example, as important constituents of highly active antibiotics of the biphenomycin type, the latter as important constituents of collagen.

Aside from some nonstereospecific syntheses, for (2S,4R)-4-hydroxyornithine the synthesis of Schmidt et al. is known (see, for example, Synthesis, 1991, p. 409; J. Chem. Soc., Chem. Commun., 1991, p. 275; Synthesis, 1992, p. 1025), which proceeds starting from enantiomerically pure, protected glyceraldehyde over 9 stages to give (2S,4R)-4-hydroxyornithine. As a consequence of the expensive reactant and the low overall yield, such a process is unsuitable for industrial realization. The process of Jackson et al., J. Org. Chem., 1992, 57, p. 3397, which starts from L-serine, also leads only in a very low overall yield to the desired product. Although some syntheses are likewise known for the homologue (2S,5R)-5-hydroxylysine (see, for example: Löhr et al., Synthesis, 1999, p. 1139), the multitude of reaction steps in this case too, and in particular the inadequate control of the stereochemistry, limits the application to the laboratory scale.

There is therefore a need, starting from favorable reactants, for a generally applicable synthetic route for preparing derivatives and homologues of 4-hydroxyornithine.

A process has now been found for preparing chiral amino acid derivatives of the general formula (I)

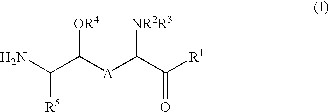

in which

R¹ is C₁-C₁₂-alkoxy, (C₁-C₁₂-alkyl)₂N—, (C₁-C₁₂-alkyl)NH— or the N-terminal end of an end group-protected amino acid or of an end group-protected peptide,

R² is a protecting group,

R³ is hydrogen, (C₁-C₁₂)-alkyl, aryl having from 6 to 10 framework carbon atoms, or C₇-C₁₃-arylalkyl or

R² and R³ together are a 1,2-dimethylenearyl radical and

R⁴ is hydrogen or

R¹ and R⁴ together are a chemical bond and

R⁵ is C₁-C₁₂-alkyl or C₇-C₁₃-arylalkyl and

A is a further substituted or unsubstituted C₁-C₄-alkylene radical, which is characterized in that compounds of the general formula (II)

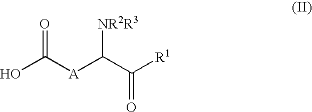

in which

R¹, R², R³ and A are as defined above

a) are converted to an activated acid derivative and then reacted with deprotonated nitro compounds which derive from the general formula (III),

R⁵—CH₂NO₂ (III)

in which

R⁵ is as defined above to give nitro ketones of the general formula (IV)

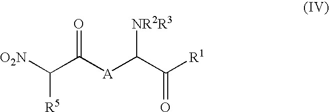

in which

R¹, R², R³, R⁵ and A are each as defined above,

b) these nitro ketones are reduced to nitro alcohols of the general formula (V)

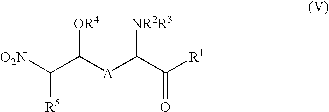

in which

R¹, R², R³, R⁴, R⁵ and A are each as defined above and

c) these nitro alcohols are reduced to the chiral amino acid derivatives of the general formula (I) in which R¹, R², R³, R⁴, R⁵ and A are each as defined above.

In this context, C₁-C₁₂-alkoxy is a straight-chain or cyclic, branched or unbranched C₁-C₁₂-alkoxy radical, for example methoxy, ethoxy, n-propoxy, isopropoxy, n-butoxy, isobutoxy, tert-butoxy, n-pentyloxy, isopentyloxy, 2,2-dimethylpentyloxy, cyclopentyloxy, cyclohexyloxy, adamantyloxy, D-methoxy or L-menthoxy.

In the contexts specified, C₁-C₁₂-alkyl is in each case independently a straight-chain or cyclic, branched or unbranched C₁-C₁₂-alkyl radical, for example methyl, ethyl, n-propyl propyl, isopropyl, n-butyl, isobutyl, n-hexyl or cyclohexyl.

In this context, the N-terminal end of an end group-protected amino acid or of an end group-protected peptide means that R¹ is an amino acid bonded via the nitrogen or a polymer composed of amino acids, whose free functionalities, for example amino groups, carboxylic acid groups or hydroxyl groups are protected by derivatization in such a way that side reactions at these fimctionalities are substantially suppressed under inventive conditions.

Such measures are sufficiently familiar to those skilled in the art, for example from T. W. Greene, P. G. Wuts, Protective Groups in Organic Synthesis, 3^{rd} edition, Wiley Interscience, 1999 and, for amino and hydroxyl groups include, for example, acylations, carbamoylations and sulfonylations, and, for carboxylic acid groups, for example, esterifications or the conversion to amides.

For R², protective groups in this context are those groups which very substantially suppress a reaction of the amino function under the inventive reaction conditions, and can be detached again in a high selectivity. Such protecting groups are known to those skilled in the art (T. W. Greene, P. G. Wuts, Protective Groups in Organic Synthesis, 3^{rd} edition, Wiley Interscience, 1999) and include, for example, protecting groups such as tert-butyloxycarbonyl, fluorenylmethyloxycarbonyl, benzyloxy-carbonyl or allyloxycarbonyl and benzyl.

For R³, aryl means aromatic radicals having from 6 to 10 framework carbon atoms, for example phenyl or naphthyl, which may be substituted by no, one, two or three further substituents from the group of C₁-C₄-alkyl or C₁-C₄-alkoxy, for example o-tolyl tolyl, m-tolyl, p-tolyl, o-anisyl, m-anisyl, p-anisyl or phenetyl.

In this context, C₇-C₁₃-arylalkyl is radicals, for example benzyl, 1-ethylphenyl, 2-ethylphenyl or p-xylyl.

In this context, 1,2-dimethylenearyl is, for example, 1,2-dimethylenephenyl.

For A, substituted or unsubstituted C₁-C₄-alkylene radicals are, for example, methylene, 1,1-ethylene, 1,2-ethylene, 1,2-propylene, 1,3-propylene, 1,3-butylene, 1,4-butylene or 2,3-butylene.

The compounds of the general formula (II) used as starting materials are either commercially available or can be prepared in accordance with existing literature or in a similar manner. The same applies to the nitro compounds of the general formula (III).

The protected amino acids of the general formula (II) used for the process according to the invention are preferably those in which

R¹ is a sterically demanding C₃-C ₁₂-alkoxy radical, for example isopropoxy, tert-butoxy, cyclopentyloxy, cyclohexyloxy, D-menthoxy, L-menthoxy or 1-adamantoxy,

R² is tert-butyloxycarbonyl (t-boc), benzyloxycarbonyl (cbz); fluorenylmethyl-oxycarbonyl (Fmoc), allyloxycarbonyl (aoc) or benzyl

R³ is hydrogen,

R⁴ is hydrogen,

R⁵ is hydrogen or methyl,

A is methylene or 1,2-ethylene.

For the process according to the invention, the protected amino acids of the general formula (II) used are more preferably those in which

R¹ is tert-butoxy

R² is tert-butyloxycarbonyl (t-boc), benzyloxycarbonyl (cbz) or fluorenyl-methyloxycarbonyl (Fmoc),

R³ is hydrogen,

R⁴ is hydrogen,

R⁵ is hydrogen and

A is methylene.

Very particular preference is given to using 1-tert-butyl N-(tert-butyloxycarbonyl)-aspartate as the protected amino acid for the process according to the invention.

The nitro compounds of the general formula (III) used are preferably nitromethane and nitroethane, more preferably nitromethane.

The free carboxylic acid groups of the protected amino acid derivatives of the general formula (II) in which R¹, R², R³ and A are each as most generally defined above can be converted to an activated acid derivative and subsequently reacted with deprotonated nitro compounds [step a)] either in separate reaction steps with isolation of the intermediates or without isolation of the activated acid derivative or of the deprotonated nitro compounds. Preference is given to carrying out step a) without intermediate isolation.

Useful activated acid derivatives include, for example, imidazolides or phenyl esters; preference is given to the imidazolides.

The preparation of nitro ketones from carboxylic acids by preparing acid imidazolides and reacting them with deprotonated nitro compounds without isolating intermediates is already known (see also: Baker, Putt, Synthesis, 1978, p. 478; Yuasa, Tsuruta, Synthetic Communications, 1998, 28(3), p. 395). WO 96/01788 also discloses the preparation of nitro ketones from the C, terminus of amino acids.

However, the yields of the nitro ketones in all cases are either low or strongly dependent on the selection of the substrate, of the solvent, of the temperature, of the amount of the activating reagent used, and of the base used for the deprotonation of the nitro compound.

To carry out step a) of the process according to the invention, preference is given to the following procedure:

1) Reacting the protected amino acid derivative of the general formula (II) with carbonyldiimidazole in a substantially anhydrous, inert solvent.

2) Deprotonating the nitro compound of the general formula (III) in a substantially anhydrous, inert solvent with a base.

3) Reacting the activated acid derivative from step 1) with the deprotonated nitro compound from step 2).

4) Working up the reaction mixture.

The amount of carbonyldiimidazole in step 1) may be, for example, from 1.0 to 1.5 equivalents based on the free carboxylic acid groups of the protected amino acid derivatives. Preference is given to from 1.05 to 1.2 equivalents.

Useful inert solvents for step 1) and step 2) include, for example: ethers such as tetrahydrofuran, diethyl ether, methyl tert-butyl ether or dioxane, or polar aprotic solvents such as dimethylformamide, dimethyl sulfoxide or N-methylpyrrolidone, a mixture of such solvents, or else be the nitro compound used itself, as long as its melting point is above 0° C.

In this context, substantially anhydrous means a water content of less than 1% by weight, preferably less than 0.03% by weight.

The amount of nitro compound in step 2) may be selected, for example, in such a way that it is from 1.0 to 100 times the free carboxylic acid groups of the protected amino acid derivative of the general formula (II). Preference is given to from 1.2 to 20 equivalents. Particular preference is given to from 2 to 10 equivalents.

Useful bases are, for example, alkali metal hydrides, hydroxides, carbonates, C₁-C₆-alkoxides, amides, substituted amides or phosphazene bases. Preference is given to the hydrides, carbonates, hydroxides, methoxides, ethoxides, tert-butoxides and diusopropylamides of lithium, sodium and potassium. Very particular preference is given to potassium tert-butoxide.

The amount of base may be selected, for example, in such a way that it is from 1.0 to 2.0 equivalents based on the free carboxylic acid groups of the protected amino acid derivatives of the general formula (II). Preference is given to from 1.05 to 1.3 equivalents. In the case of bases which are insoluble or only sparingly soluble in the solvent, a large excess (up to 500 equivalents) of base is generally uncritical. The base may be used in dissolved, solid or suspended form. It may be initially charged or added to the solution of the nitro compound.

The temperature in the preparation of the activated acid derivative in step 1) may be, for example, from 0 to 80° C., preferably from 15 to 25° C.

The reaction time in step 1) may be, for example, from 30 min to 24 h, preferably from 3 to 8 h.

The temperature in the deprotonation of the nitro compound in step 2) may be, for example, from −20° C. to 25° C., preferably from −5 to 5° C. The reaction time in step 2) may be, for example, from 5 min to 24 h, preferably from 30 min to 1 h.

The temperature in the reaction of the activated acid derivative with the deprotonated nitrogen compound in step 3) may be, for example, from 0 to 80° C., preferably from 15 to 25° C. The reaction time in step 3) may be, for example, from 4 h to 24 h, preferably from 8 to 16 h.

The activated acid derivative may be reacted with the deprotonated nitro compound, for example, in such a way that the reaction mixture from step 1) is added to the reaction solution from step 2) or vice versa. Preference is given to adding the activated acid derivative from step 1) to the deprotonated nitro compound from step 2).

The reaction mixture from step 3) may be worked up, for example, in such a way that water and an acid or an aqueous acid solution are added, and extraction is then effected with a water-immiscible or only sparingly water-miscible solvent, and the water-immiscible or only sparingly water-miscible solvent is then removed. This may be effected, for example, by distillation.

The amount of the acid used should generally be selected in such a way that it corresponds to or exceeds the amount of the amount of base used in step 2). Suitable acids or aqueous acid solutions are, for example, dilute mineral acids such as hydrochloric acid or sulfuric acid, carboxylic acids such as acetic acid or citric acid. In this context, dilute means a molar concentration of 2 mol/l.

Preference is given to using 1 molar aqueous hydrochloric acid.

Suitable water-imiscible or only sparingly water-miscible solvents for the extraction are, for example:

ethers such as diethyl ether, methyl tert-butyl ether, esters such as ethyl acetate, butyl acetate, chlorinated hydrocarbons such as chloroform or dichloromethane, aromatic solvents such as toluene or xylenes, hydrocarbons such as hexane or heptane, and also mixtures of such solvents.

For the reduction of nitro ketones to the corresponding nitro alcohols, processes are known which use boron-hydrogen and aluminum hydrogen compounds (see, for example, WO 96/01788).

Likewise suitable for step b), the preparation of nitro alcohols of the general formula (TV), are the boranes specified in the literature, for example borane, diisoamylborane, 9-bora-bicyclo[3.3.1]nonane, borohydrides such as sodium borohydride, lithium borohydride, lithium triethylborohydride and lithium tri(sec-butyl)borohydride and aluminohydrides such as lithium tri(tert-butoxy)aluminum hydride, which can be used by the customary processes known to those skilled in the art. The diastereoselective reduction of nitro ketones has already been described in the literature (see, for example, Caille, J.-C.; Bulliard, M.; Laboue, B.; Asymmetric reduction of prochiral ketones, in Chirality Ind. II, Collins, A.N.; Sheldrake, G.N.; Crosby, J. (Eds.), Wiley, Chichester, UK 1997, p. 391-401).

For a highly diastereoselective reduction, preference is given in the process according to the invention to using lithium tri(sec-butyl)borohydride or a solution thereof. The reaction temperature in the reduction may be, for example, from −90 to 0 C., preferably from −80 to −60° C.

Step c), which includes the reduction of nitro alcohols to the corresponding amino alcohols of the general formula (I) can be carried out in a similar manner to literature methods, for example catalytically in the presence of a hydrogen source.

Suitable catalysts may be, for example:

palladium/carbon, rhodium/carbon, Raney nickel or platinum black.

Suitable hydrogen sources are, for example, hydrogen and also hydride transfer reagents, for example formic acid, sodium formate and ammonium formate.

Preference is given to reducing all the palladium/carbon in the presence of ammonium formate.

In the inventive manner, chiral amino acid derivatives of the general formula (I) are obtained.

Steps b) and c) can be carried out not only sequentially but also simultaneously when conditions are employed which can reduce both nitro groups and ketones. Such conditions may be, for example, the hydrogenation over ruthenium complexes and/or palladium complexes in the presence of hydrogen (see, for example, also Y. Yuasa et al., Synth. Commun. 1998, 28, p. 395).

However, preference is given to the sequential reduction.

These chiral amino acid derivatives are suitable in particular for further use, for example, in a process for preparing antibiotics of the biphenomycin type, for example biphenomycin A and biphenomycin B.

The particular advantage of the process according to the invention is based on the fact that for the preparation of derivatives and homologues of (2S,4R)-4-hydroxyornitine now entails only 3 reaction stages starting from readily obtainable, protected amino acids. These reaction stages proceed in high yields and in good to very good overall optical yields.

### EXAMPLES

### Example 1

### a) Preparation of tert-butyl N-(tert-butoxycarbonyl)-5-nitro-4-oxo-L-norvalinate

1.0 g (3.46 mmol) of tert-butyl N-(tert-butyloxycarbonyl)-1-aspartate is added at RT to a solution of 0.59 g (3.63 mmol) of 1,1′-carbonyldiimidazole in 50 ml of anhydrous THF. This mixture is left to stir for a further 5 h. In parallel, a solution of 1.87 ml (34.6 mmol) of nitromethane in 20 ml of anhydrous THF is added dropwise at 0° C. to a solution of 0.43 g (3.80 mmol) of t-BuOK in 50 ml of dry THF and stirred for 1 h. The solution of the activated acid, tert-butyl N-(tert-butyloxycarbonyl)-l-asparatate, is then added dropwise to this solution. After the addition, the reaction mixture is stirred for a further 15 h. Subsequently, the solution is treated with 1 M HCl, extracted with ethyl acetate (2×), washed with saturated aqueous NaCl solution and then dried over MgSO₄. The solvent is removed under reduced pressure. No fuirther purification step is required.

Yield: 1.10 g (96%), white solid. m.p.=78-82° C. ¹H NMR (400 MHz, CDCl₃): δ=5.35 (d, J=6.6 Hz, 1H, NH), 5.23 (s, 2H, CH₂NO₂), 4.39 (m, 1H, CHNH), 3.03 (m, 2H, CH₂), 1.38 (s, 18H, t-Bu). ¹³C NMR (100 MHz, CDCl₃): δ=194.3, 169.3, 155.5 (C═O), 83.3 (CH₂—NO₂), 83.0+82.5 (t-Bu), 50.2 (CH—NH), 42.9 (CH₂), 28.31, 28.27 (CH₃). MS (ESI): m/z=333.2 [M+H]⁺. FT-ICR-MS: m/z ([M+Na]⁺)=355.14757 (calc.), 355.14750 (found). [α]_{D}²⁵=−16 (ethanol:water=95:5 (v/v), c=1).

### b) Preparation of tert-butyl (2S,4R)-2-[(tert-butoxycarbonyl)amino]-4-hydroxy-5-nitropentanoate

0.67 mg (2.00 mmol) of the nitro ketone from a) are dissolved in 30 ml of anhydrous THF and cooled to −78° C. 2 ml of a 1 M solution of L-Selectride in THF are then added dropwise and the temperature is maintained. The end of the reaction is determined by TLC (eluent=toluene:THF:ethyl acetate, 90:5:5). After approx. 3 h, the reaction has ended and the reaction solution is quenched with saturated aqueous NH₄Cl solution. The aqueous phase is extracted with ethyl acetate (3 ×). The crude product exhibits a diastereomeric ratio of 85:15 (HPLC) in favor of the desired (2S,4R) isomer. The crude product is purified by column chromatography on silica gel using the eluent toluene:THF:ethyl acetate (90:5:5) (R_{f=0.18}). At a diastereomeric enrichment of >90:10, it is alternatively also possible for complete purification to recrystallize from hexane.

Yield: 0.28 g (42%), white solid m.p:=132.5-134.5° C. ₁H NMR (400 MHz, CDCl₃): δ=5.43 (d, J=7.5 Hz, 1H, NH), 4.59-4.25 (m, 4H, CH—OH, CH—NH, CH₂—NO₂), 3.38 (d, J=3.7 Hz, 1H, OH), 2.09 (m, 1H, CH₂), 1.89 (m, 1H, CH₂), 1.47 (s, 9H, t-Bu), 1.46 (s, 9H, t-Bu). ¹³C NMR (100 MHz, CDCl₃): δ=171.0 (C═O), 83.0 (CH₂—NO₂), 80.6 +80.1 (O—C(CH₃)₃), 66.1 (CH—OH), 51.3 (CH—NH), 36.9 (CH₂), 28.3 +28.1 (C(CH₃)₃. MS (ESI): m/z=335 [M+H]⁺. FT-ICR-MS: m/z ([M+Na]⁺)=357.16322 (calc.), 357.16329 (found). [α]_{D}²⁵ =−5.025 (ethanol/water=95:5 (v/v), c=1).

### c) Preparation of tert-butyl (2S,4R)-N^{α}-(tert-butoxycarbonyl)-4-hydroxy-ornithinate

The nitro alcohol from b) (5 g, 14.9 mmol) is dissolved in 50 ml of methanol. The reaction mixture is cooled to −10° C. and palladium on carbon (10%, purissimum, Fluka) (2.5 g) and dry ammonium formate (9.43 g, 150 mmol, 10 eq) are added with stirring (reaction temperature at −10° C.). After stirring for 2 h, the catalyst is filtered off. The solvent is removed and ethyl acetate and sat. NaHCO₃ solution are added (pH≧7). After phase separation and two additional washings with ethyl acetate, the combined organic phases are washed with sat. NaCl solution and dried over Na₂SO₄, and the solvent is removed under reduced pressure. The product is obtained as a clear, light yellowish oil in a yield of 4.55 g (100%). As a consequence of its instability, the product 3 is subjected to no further purification step and converted directly to compound 4.

¹H NMR (400 MHz, CDCl₃): δ=7.94 (s (br), 2H, NH₂), 5.56 (m, 1H, NH), 4.29 (m, 1H, CHOH), 4.25 (m, 1H, OH), 4.11 (m, 1H, CHNH), 3.02 (m, CH₂—NH₂0, 2.85 (m, 1H, CH₂—NH₂), 1.92 (m, 1H, CH₂), 1.85 (m, 1H, CH₂), 1.46 (s, 9H, t-Bu), 1.44 (s, 9H, t-Bu). ¹³C NMR (100.58 MHz, CDCl₃): δ=66.5 (CHOH), 59.5 (CH₂NH₂), 52.7 (CHNH), 38.2 (CH₂), 28.7 (CH₃). MS (ESI): m/z=305 [M+H]⁺. FT-ICR-MS: m/z ([M+H]⁺)=305.20710 (calc.), 305.20709 (found).

### d) Preparation of tert-butyl (2S, 4R)-N^{α}(tert-butyloxycarbonyl)-N^{δ}-(benzyloxy-carbonyl)-4-hydroxyornithinate

The amino alcohol from c) (4.55 g of crude product, ˜15 mmol) is dissolved in 50 mL of DMF, and N,N-diisopropylamine (3.8 g, 30 mmol, 2 eq) and benzyl N-succinimidylcarbonate (Z-OSu) (3.74 g, 15 mmol) are added. After a reaction time of 16 h, the solvent is removed under reduced pressure, and the residue is taken up in dichloromethane and washed in succession with 5% aqueous citric acid, sodium hydrogencarbonate solution and sat. sodium chloride solution. After drying over sodium sulfate and removing the solvent under reduced pressure, the product is purified by silica gel chromatography with hexane/ethyl acetate (10:1=>3:1).

Yield (over two stages): 3.3 g (51%), white solid m.p: 79-81° C. ¹H NMR (400 MHz, CDCl₃): δ=7.32 (m, 5H, aromat.), 5.49 (m, 2H, NH, OH), 5.09 (s, 2H, Ph-CH₂), 4.22 (m, 1H, CH—OH), 3.89 (m, 1H, CH—NHBoc), 3.35 (m, 1H, CH₂N), 3.11 (m, 1H, CH₂N), 1.92 (m, 1H, C—CH₂—C), 1.45 (s, 9H t-Bu), 1.42 (s, 9H, tBu). ¹³C NMR (100.58 MHz, CDCl₃): δ=172.1, 157.5, 156.2 (C═O), 136.8, 128.9, 128.7, 128.5 (ar.), 82.7, 80.5 (tBu), 69.1 (C—OH), 67.2 (CH₂Ph), 52.5 (CHN), 47.2 (CH₂N), 38.0 (C—CH₂—C), 28.7, 28.3 (CH₃). FT-ICR-MS: m/z ([M+H]⁺=4.39.24388 (calc.), 439.24427 (found).

1. A process for preparing chiral amino acid derivatives of the general formula (I) 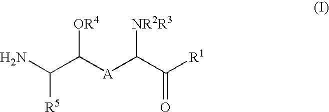
in which
R¹ is C₁-C₁₂-alkoxy, (C₁-C₁₂-alkyl)₂N—, (C₁-C₁₂-alkyl)NH— or the N-terminal end of an end group-protected amino acid or of an end group-protected peptide,
R² is a protecting group,
R³ is hydrogen, (C₁-C₁₂)-alkyl, aryl having from 6 to 10 framework carbon atoms, or arylalkyl having from 7 to 12 carbon atoms or
R² and R³ together are a 1,2-dimethylenearyl radical and
R⁴ is hydrogen or
R¹ and R⁴ together are a chemical bond and
R⁵ is C₁-C₁₂-alkyl or C₇-C₁₃-arylalkyl and
A is a further substituted or unsubstituted C₁-C₄-alkylene radical, comprising
a) converting compounds of the general formula (II) 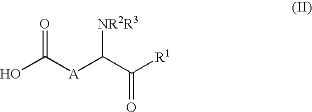
in which
R¹, R², R³ and A are as defined above;
to an activated acid derivative and then reacting the activated acid derivative with deprotonated nitro compounds which derive from the general formula (III),
R⁵—CH₂NO₂ (III)
in which
R⁵ is as defined above to give nitro ketones of the general formula (IV) 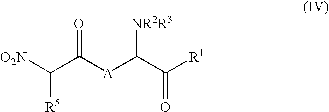
in which
R¹, R², R³, R⁵ and A are each as defined above,
b) reducing these nitro ketones to nitro alcohols of the general formula (V) 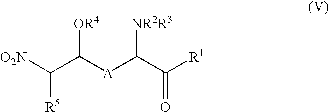
in which
R¹, R², R³, R⁴, R⁵ and A are each as defined above and
c) reducing these nitro alcohols to the chiral amino acid derivatives of the general formula (I) in which R¹, R², R³, R⁴, R⁵ and A are each as defined above.

2. The process of claim 1, characterized in that the conversion to a nitroketone of step a) comprises the following steps:
1) converting the compounds of the general formula (II),
in which
R¹ is C₁-C₁₂-alkoxy, (C₁-C₁₂-alkyl)₂N—, (C₁-C₁₂-alkyl)NH— or the N-terminal end of an end group-protected amino acid or of an end group-protected peptide,
R² is a protecting group
R³ is hydrogen, (C₁-C₁₂)-alkyl, aryl having from 6 to 10 framework carbon atoms, or arylalkyl having from 7 to 12 carbon atoms or
R² and R³ together are a 1,2-dimethylenearyl radical and
R⁴ is hydrogen or
R¹ and R⁴ together are a chemical bond and
R⁵ is C₁-C₁₂-alkyl, or C₇-C₁₃-arylalkyl and
A is a further substituted or unsubstituted C₁-C₄-alkylene radical with carbonyidiimidazole in from 1.0 to 1.5 equivalents based on free carboxylic acid groups in a solvent.
2) at least partially deprotonating from 1.0 to 100 equivalents of a nitro compound of the general formula (III) in which R⁵ is as defined in claim 1 with from 1.0 to 2.0 equivalents of a base, the amounts specified relating to the amount of the free carboxylic acid groups of the compounds of the general formula (II) in step 1).
3) reacting the reaction mixture from 1) with the reaction mixture from 2).

3. The process of claim 2, characterized in that the temperature for step 1) is from 0 to 80° C.

4. The process of claim 2, characterized in that the temperature for step 2) is from −20 to 25° C.

5. The process of claim 2, characterized in that the base for step 2) is selected from the group consisting of the hydrides, hydroxides, carbonates, C₁-C₆-alkoxides, amides and organic amides of lithium, sodium and potassium.

6. The process of claim 1, characterized in that compounds are used in which
R¹ is isopropoxy or tert-butoxy,
R² is tert-butyloxycarbonyl, fluorenylmethyloxycarbonyl, benzyloxycarbonyl or allyloxycarbonyl,
R³ is hydrogen,
R⁴ is hydrogen,
R⁵ is hydrogen or methyl,
A is methylene.

7. The process of claim 1, characterized in that the reduction of the nitro ketones of step b) is carried out diastereoselectively.

8. The process of claim 1, characterized in that the nitro ketones are reduced in step b) with lithium tris(isobutyl)borohydride.

9. The process of claim 1, characterized in that the nitro group is reduced in step c) by catalytically reducing it in the presence of a hydrogen source selected from the group consisting of hydrogen, formic acid, sodium formate or ammonium formate.

10. Compounds of the general formula (IV) 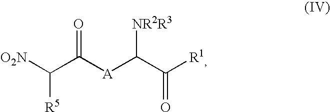
in which
R¹ is C₁-C₁₂-alkoxy, (C₁,-C₁₂-alkyl)₂N—, (C₁-C₁₂-alkyl)NH— or the N-terminal end of an end group-protected amino acid or of an end group-protected peptide,
R² is a protecting group,
R³ is hydrogen, (C₁-C₁₂)-alkyl, aryl having from 6 to 10 framework carbon atoms, or arylalkyl having from 7 to 12 carbon atoms or
R² and R³ together are a 1,2-dimethylenearyl radical and
R⁴ is hydrogen or
R¹ and R⁴ together are a chemical bond and
R⁵ is C₁-C₁₂-alkyl or C₇-C₁₃-arylalkyl and
A is a substituted or unsubstituted C₁-C₄-alkylene radical.

11. tert-Butyl N-(tert-butoxycarbonyl)-5-nitro4-oxo-L-norvalinate.

12. Compounds of the general formula (V) 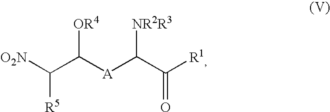
in which
R¹ is C¹-C₁₂-alkoxy, (C₁-C₁₂-alkyl)₂N—, (C₁-C₁₂-alkyl)NH— or the N-terminal end of an end group-protected amino acid or of an end group-protected peptide,
R² is a protecting group,
R³ is hydrogen, (C₁-C₁₂)-alkyl, aryl having from 6 to 10 framework carbon atoms, or arylalkyl having from 7 to 12 carbon atoms or
R² and R³ together are a 1,2-dimethylenearyl radical and
R⁴ is hydrogen or
R¹ and R⁴ together are a chemical bond and
R⁵ is C₁-C₁₂-alkyl or C₇-C₁₃-arylalkyl and
A is a further substituted or unsubstituted C₁-C₄-alkylene radical.

13. (2S,4R)-2-tert-butyl [(tert-butyloxycarbonyl)amino]-4-hydroxy-5-nitro-pentanoate.

14. A process for preparing 5-hydroxylysine or 4-hydroxyornithine, or derivatives of 5-hydroxylysine or 4-hydroxyornithine comprising providing the compounds of claim 10.

15. A process for preparing 5-hydroxylysine or 4-hydroxyornithine, or derivatives of 5-hydroxylysine or 4-hydroxyornithine comprising providing the compounds of claim 12.

16. A process for preparing biphenomycins comprising incorporating compounds of claim 10.

17. A process for preparing biphenomycins comprising incorporating compounds of claim 12.

18. Biphenomycins, characterized in that they are prepared by a process of claim 16.

19. Biphenomycins. characterized in that they are prepared by a process of claim 17.
